(19) United States
(12) Patent Application Publication
(10) Pub. No.: US 2010/0143396 A1
(43) Pub. Date: Jun. 10, 2010

(54) Method for Preparing a Covalently Cross Linked Oligomer of Amyloid Beta Peptides

(76) Inventors: Karen M. Grimm, Duryea, PA (US); Joseph G. Joyce, Lansdale, PA (US); Xiaoping Liang, Collegeville, PA (US); Denise Nawrocki, Annandale, NJ (US)

Correspondence Address: MERCK, P O BOX 2000, RAHWAY, NJ, 07065-0907, US

(21) Appl. No.: 11/922,552

(22) PCT Filed: Jun. 26, 2006
(86) PCT No.: PCT/US2006/024742

§ 371 Date: Dec. 18, 2007

Related U.S. Application Data
(60) Provisional application No. 60/695,528, filed on Jun. 30, 2005.

(51) Int. Cl. A61K 39/385; C07K 1/00; C07K 1/16; A61P 25/28

(52) U.S. Cl. 424/193.1; 530/345; 530/344

(57) ABSTRACT

The invention relates to a method for the preparation of a stabilized cross-linked oligomer of amyloid beta using a near-zero length bifunctional cross-linking agent for use as an immunogen for the generation of antibodies for the treatment of Alzheimer's Disease and other conditions related to abnormal amyloid beta aggregation. A preferred bifunctional cross-linking agent is 1,5-difluoro-2,4-dinitrobenzene (DFDNB).

### FIELD OF THE INVENTION

The invention relates to a composition and method for the preparation of stabilized cross-linked oligomers of amyloid beta for use as an immunogen for the generation of antibodies for the treatment of Alzheimer's disease and other conditions related to abnormal amyloid beta aggregation.

### BACKGROUND OF THE INVENTION

Peptides derived from human amyloid precursor protein (APP), i.e. amyloid-beta (Aβ) peptides, undergo self-association in aqueous solution to form a complex and heterogeneous mixture of oligomeric forms (Klein, W. L., et al., Trends Neurosci. 24: 219-224 (2001)). One class of such oligomers, known as amyloid-derived diffusible ligands (ADDLs), has been produced from synthetic Aβ₄₂ peptides (Chromy, B A., et al., Biochemistry 42: 12749-12760 (2003). These soluble oligomers are reported to be associated with the neurotoxic events and symptoms which are collectively known as Alzheimer's disease (AD), presumably through an as yet unidentified receptor-mediated pathway (Klein, W. L., Neurochem. Int. 41: 345-352 (2002).

ADDLs, in the published literature, have been characterized in a variety of ways, but most commonly by migration patterns in gel electrophoresis and by atomic force microscopy (AFM). On SDS-PAGE gels, characteristic species are observed corresponding to monomer, dimer, trimer, and occasionally higher mass oligomers from synthetic preparations of Aβ₄₂ peptide. Similar results have been observed in brain extracts from animals in AD disease models. AFM clearly differentiates spherical, soluble oligomers from extended fibrillar and protofibrillar forms.

It has been generally recognized that Aβ peptide preparations are dynamic, i.e., they continually undergo association and dissociation events which ultimately result in higher order aggregates. The epidemiological association of amyloid plaque morphology in the post-mortem brains of AD patients has led to the theory that disease is correlated with the deposition of amyloid fibrils. However, recent studies have suggested that the relevant neurotoxic and cognitive-impairing events are mediated by soluble oligomers, or ADDLs, in the absence of plaque deposition (Kirkitadze, M. D., et al., J. Neurosci. Res.: 69:567-577 (2002)). The major peptide species in brain, Aβ₄₀ and Aβ₄₂, demonstrate markedly different propensities for aggregation, with Aβ₄₀ existing largely in a monomeric state. As the elevation of Aβ₄₂ levels in transgenic animal models correlates with increased AD-like manifestations, it is believed that self-association of Aβ₄₂ is the causative event for neurotoxicity. In vitro studies with synthetic peptides have confirmed the aggregation-prone nature of Aβ₄₂ relative to Aβ₄₀ (Jarrett, J. T., et al., Ann. NY Acad. Sci.: 695:144-148 (1993)). Since monomeric Aβ species are present in normal tissue, they are considered self-antigens which are generally not recognized by the immune system. Self-association in the presence of elevated Aβ₄₂ concentrations leads to oligomer formation, and while the structural form of these oligomers is not known at the present time, the possibility exists that the interaction of two or more monomeric peptides can produce novel conformational epitopes (neo-epitopes) which are no longer recognized as self by the immune system. As such, an antibody response specifically directed toward such conformational epitopes might be protective in either active or passive immunological approaches to AD treatment.

The observation that defined preparations of synthetic amyloid peptides contain toxic oligomers (Klein, W. L., et al., Neurobiol. Aging: 25:569-580 (2004)) allows for a method for production of immunogens to achieve a directed antibody response. However, if used “as is,” the resulting preparations may be sub-optimal for achieving the desired antibody response: (1) the instability of such preparations makes it difficult to define and target an oligomer population of desired size, since the system is in a state of flux, (2) the presence of appreciable levels of monomer may have a detrimental effect, since this species is recognized as “self” and large quantities may prevent recognition of novel conformational epitopes on oligomers, and (3) high levels of monomer may induce a response directed toward epitopes shared between monomer and oligomer. In this latter scenario, the antibody response may be directed to an immuno-dominant epitope in the sense that the majority of antibodies produced all recognize the same region of the molecule. Such may be the case for the Aβ peptide since several monoclonal antibodies recognizing the amino terminus of the molecule (residues 1-15) have been reported. While this may not necessarily be detrimental, the safety level of such passively administered antibody formulations would be enhanced if they recognized only the toxic oligomer component and not the monomeric self antigen. Indeed, Pfeifer at al. reported that passive administration of a monoclonal IgG antibody recognizing residues 2-6 of Aβ resulted in a significant reduction of diffuse amyloid burden, but also led to a 2-fold increase in cerebral amyloid angiopathy-associated hemorrhage (Pfeifer, M., et al., Science 298: 1379 (2002)).

The invention herein is directed to an improved method for generating covalently stabilized oligomeric forms of Aβ-derived peptides, substantially free of monomeric Aβ peptides, for use as immunogens for the generation of antibodies to treat AD.

### SUMMARY OF THE INVENTION

One embodiment of the invention describes a method for production of covalently coupled oligomeric forms of Aβ-derived peptides. The peptides include full length Aβ, which comprises amino acid residues 1-40 (Aβ₄₀) (SEQ ID NO. 1) or amino acid residues 1-42 (Aβ₄₂) (SEQ ID NO. 2), as well as substituted or truncated versions thereof (SEQ ID NOS. 3-7). The peptide source may be synthetic, natural, or produced by recombinant technologies. Such a method provides an oligomeric form that is stabilized and has a predominant oligomeric species with a mass range of 100 kDa to 200 kDa.

In still another embodiment of the invention, the method provides a composition comprising an immunogen having a conformational epitope of a soluble, oligomer species of Aβ₄₂.

In another embodiment of the invention, a method is provided for a process for purifying a covalently coupled oligomeric form of Aβ-derived peptides such that populations of defined molecular weight (Mw) can be generated. This embodiment comprises the use of a chromatographic separation step in which populations are separated based on mass, a technique referred to as size exclusion chromatography (SEC).

### DETAILED DESCRIPTION OF THE INVENTION

Cross-linking is an established technique used for stabilizing oligomeric structures and for increasing the immunogenicity of a peptide preparation. One common approach utilizes chemical cross-linking agents, such as glutaraldehyde, (LeVine, H., Neurobiol. Aging, 16:755-764 (1995)). This approach has been used for the determination of the subunit structure of oligomeric proteins, but it has several drawbacks when applied to peptides. Glutaraldehyde is not a zero-length cross-linker and, as such, it introduces additional linker atoms within the structure of the cross-linked species, which may perturb the native structure. For Aβ₄₂ oligomers this can result in cross-linking between adjacent oligomers rather than within a given oligomer (Bitan, G., et al., J. Biol. Chem. 276: 35176-35184 (2001)). Glutaraldehyde also contributes to a number of undesirable side reactions such as self-polymerization and lysine modifications under certain conditions. Id. More recent reports describe the use of photo-induced cross-linking of unmodified proteins (PICUP) in Aβ oligomerization studies (Bitan, G., et al., J. Biol. Chem. 276: 35176-35184 (2001); Bitan, G., et al., Proc. Natl., Acad. Sci. USA 100: 330-335 (2003); Bitan, G., et al., J. Biol. Chem. 278: 34882-34889 (2003); and Bitan, G. and Teplow, D. B., Acc. Chem. Res. 37: 357-364 (2004)). The PICUP methodology, as shown in U.S. Pat. No. 6,613,582, utilizes a light-induced free radical mechanism for covalently linking amino acid residues (primarily aromatics, His, or Met) which are in close proximity in the structure. While it utilizes relatively short reaction times, the method is highly sensitive to the parameters of light input and intensity. Further, volume and concentration changes may have dramatic effects on the final product composition, such as altering the proportion of lower order oligomer and monomer species present. The reports to date have shown that the species generated are generally of lower order, i.e. dimer through octomer. Id.

The invention described herein comprises a method for producing a covalently coupled oligomeric form of Aβ-derived peptides using the near-zero-length bifunctional cross-linking agent, 1,5-difluoro-2,4-dinitrobenzene (DFDNB), that is specifically isolated by size exclusion chromatography. The oligomeric species, prepared according to the protocol set forth in Example 1, has an average molecular mass between 100 kDa and 200 kDa and has minimal monomer contamination, that is, the percentage of unincorporated monomer is less than 5%.

DFDNB reacts with primary amines via its two fluorine substituents by nucleophilic aromatic substitution to give an aryl amine derivative. The choice of a near-zero-length reagent is important for cross-linking self-associating peptide species for several reasons: (1) it insures that only residues which are already in close proximity, i.e., those residues that are close in the native structure, will react, (2) such a reagent minimizes the introduction of extraneous spacer atoms into the structure which may have a destabilizing or conformation-altering effect, and (3) the small, hydrophobic nature of the reagent can allow better penetration into the hydrophobic interior of the peptide oligomer. In the instant matter, DFDNB was chosen for Aβ cross-linking since the spanning distance between linked atoms was less than 5 Å and the rigidity of the cross-link was considerably higher than for other amine-reactive reagents (Green, N. S., et al., Protein Sci. 10:1293-1304 (2001)). While other zero or near-zero cross-linking agents, such as 1-ethyl-3-(3-dimethylaminopropyl) carbodiimide (EDC) and 4-phenyl-1,2,4 triazoline-3,5-dione (PTD), are known and may be employed for cross-linking peptides such as Aβ, Applicants found that neither agent provided an oligomeric species that had the optimal size and minimal contamination of the claimed method as did DFDNB.

Chemical cross-linking provides a further advantage over photo-induced cross-linking in the degree of control and ability to scale-up the process attainable with the former. Although constitutively reactive, the extent of reaction obtained with bifunctional reagents can be modified by variations in concentration, reaction time, and temperature. For photo cross-linking the primary parameters are light intensity and duration which are more difficult to control and scale in a reproducible manner. Further, the free radical mechanism inherent in photo-cross-linking can have undesirable effects on peptide structure, particularly at the longer reaction times required to generate higher mass species (Bitan, G., J. Biol. Chem. 276: 35176-35184 (2001)). As such, one of skill in the art would not necessarily look to use DFDNB for cross-linking of the amyloid-derived peptides herein.

The Aβ-derived peptides of the invention include any peptide derived from full length Aβ, which includes Aβ forms comprising a length of 39 to 43 amino acids. The full length sequence of Aβ was described in Kang et al., Nature 325:773-776 (1987). Aβ-derived peptides of the invention include, but are not limited to peptides comprising residues 1-42 (Aβ₄₂) or residues 1-40 (Aβ₄₀), as well as peptides comprising one or more mutations from the wild type sequence, conservative substitutions or alterations or truncated versions thereof. The sequence may contain natural or non-naturally occurring amino acids and may include end terminal modifications such as (1) acetylation or amidation introduced for purposes of in vivo stabilization, (2) the introduction of reactive groups for chemical conjugation, including but not limited to, cysteinylation, maleimidation, and bromoacetylation, and (3) the introduction of spacer linkages including, but not limited to, aminohexanoic acid, polyethylene glycol derivatives, and polyacidic or polybasic amino acid repeats. The Aβ-derived peptides may be synthetic, naturally derived, or produced by recombinant technologies known to those of ordinary skill in the art. The following are examples of Aβ-derived peptide sequences that may be employed within the claimed methods.

Human Aβ₄₀ (1-40):

(SEQ ID NO. 1)

DAEFRHDSGYEVHHQKLVFFAEDVGSNKGAIIGLMVGGVV
Human Aβ₄₂ (1-42):

(SEQ ID NO. 2)

DAEFRHDSGYEVHHQKLVFFAEDVGSNKGAIIGLMVGGVVIA
Human N-cysteinylated Aβ₄₂ (1-42):

(SEQ ID NO. 3)

Ac-C-Aha-DAEFRHDSGYEVHHQKLVFFAEDVGSNKGAIIGLMVGGVV
IA
Human N-maleimidated truncated Aβ (9-42):

(SEQ ID NO. 4)

maleimide-Aha-GYEVHHQKLVFFAEDVGSNKGAIIGLMVGGVVIA
Human C-maleimidated truncated Aβ (9-42):

(SEQ ID NO. 5)

GYEVHHQKLVFFAEDVGSNKGAIIGLMVGGVVIA-Aha-Lys-
maleimide
Human N-maleimidated modified Aβ (8-1(inverted);
9-42):

(SEQ ID NO. 6)

maleimide-Aha-SDHRFEADG YEVHHQKLVFFAEDVGSNKGAIIGLM
VGGVVIA
Human C-maleimidated modified Aβ (8-1(inverted);
9-42):

(SEQ ID NO. 7)

SDHRFEADGYEVHHQKLVFFAEDVGSNKGAIIGLMVGGVVIA-Aha-
Lys-maleimide

In all of the aforementioned sequences, Aha means “6-aminohexanoic acid”, Ac means “N-terminal acetylated,” N means “N-terminal” and C means “C-terminal.”

The method of the invention claimed herein is carried out such that the resulting chemical bonds between cross-linked Aβ-derived peptides are covalent in nature and irreversible under normal physiological conditions. The terms “covalently coupled” and “covalently stabilized” refer to such species which have been subject to the cross-linking method of the claimed invention and will not revert to monomer form under normal physiological conditions. Cleavage of such bonds normally requires harsh conditions such as acid or base hydrolysis at high temperatures, for example, 6 N HCl, 100° C., 20 hours). This is in contrast to the non-covalent, self-associating species which the Aβ peptides are known to adopt.

The method of the invention herein for the production of a covalently stabilized oligomer using DFDNB as the bifunctional cross-linking agent is preferred over the methods of the prior art in that the inventive method will allow for production of oligomeric species containing a broader mass range than previously reported and will allow for a method whereby monomeric species are removed from the resultant oligomeric preparation. The predominant form produced from the method herein has a molecular mass following size exclusion chromatographic fractionation in the range of 100 kDa to 200 kDa as measured by denaturing gel electrophoresis and has less than 5% monomer contamination as evidenced by gel densitometry.

In another embodiment of the invention, the method of producing a covalently stabilized oligomer by means of cross-linking an Aβ-derived peptide with a zero or near-zero length bifunctional cross-linking agent comprises a method of producing an immunogen having an oligomer-preferring epitope of the oligomeric Aβ species. Without wishing to be bound by any theory, the immunogen so produced when utilized for immunization in a mammal will provide antibodies that will be more directed to the form of Aβ responsible for its neurotoxic effects.

In still another embodiment, the invention comprises a method for purifying the cross-linked species such that populations of defined molecular weight (Mw) can be generated. Control of the cross-linking reaction can, to some extent, limit the mass distribution of species obtained, but it would not be feasible to drive the reaction toward consumption of all monomer while maintaining control of the intermediate mass species. At the extended reaction times required to effect conversion of all monomer to oligomer, the percentage of cross-linked material above 200 kDa would rise significantly. By employing size fractionation, the cross-linking can be terminated at a time which is optimal for generation of intermediate mass oligomers, and residual contaminating monomer is purified away. This process consists of a chromatographic separation step in which populations are separated based on mass, a technique referred to as size exclusion chromatography (SEC). The main advantage of this method is that more stringent separation conditions can be employed for covalently cross-linked oligomers as compared to non-covalently associating species. It is recognized that by the inherent nature of the amyloid peptides, cross-linked species may themselves undergo a degree of self-association. However, these species can be separated under dissociating conditions which include, but are not limited to, use of chemical denaturants, use of biological detergents, use of elevated temperature, and use of solvents, alone or in combination. Such treatments are expected to disrupt non-covalent interactions but will not disrupt the covalent cross-linkages. It is recognized that other chromatographic separation techniques including, but not limited to, ion exchange, reversed phase, and hydrophobic interaction can be utilized to similar ends. In a preferred embodiment of the invention, the SEC method comprises the use of a 1:1 mixture of acetonitrile and an aqueous buffer and a temperature of about 45° C. to 48° C.

The covalently stabilized oligomer of the claimed method may be used for the preparation of peptide antigen formulations as immunogens for the generation of unique and novel antibodies. Covalent cross-linking of self-associating peptide oligomers is expected to stabilize those species in a defined structure so that putative neo-epitopes not found on the monomeric peptide can be presented. When these cross-linked oligomers are used as immunogens in mice or other mammalian species, monoclonal antibodies (mAb) can be produced from the hyperimmune sera by standard methodologies and screened for oligomer-reactive specificity. These mAbs would form the basis of a passive therapeutic treatment regimen for amyloidosis which specifically targets soluble oligomers of the Aβ peptides. More specifically, the mAbs produced by these immunogens could be used as part of a treatment regime for AD. For passive immunization formulations, purified cross-linked oligomers would be filter-sterilized, formulated with an appropriate immune-stimulating adjuvant of choice, for example, aluminum hydroxyphosphate or a saponin-based adjuvant such as ISCOMATRIX®, CSL Ltd., Parkville, Australia. Those skilled in the art would know how to make such vaccine formulations. If successful in animal trials, the method of the invention could be performed under cGMP guidelines to prepare materials for human clinical trials.

The invention claimed herein provides an improved means for generating covalently-stabilized oligomeric forms of Aβ-derived peptides which can be isolated, i.e. free of monomer, and used for animal immunizations. Although the known method of photo-induced cross-linking of unmodified proteins (PICUP) has been demonstrated to provide effective cross-linking of ADDLs (Bitan, G. J., Biol. Chem. 276: 35176-35184 (2001)), the reports to date have shown that the species generated are generally of lower order, i.e. dimer through approximately octamer. Furthermore, gel analyses have shown that a significant proportion of monomer is still present in these preparations. Whereas the quantity of material generally required for immunologic animal studies is appreciable, the previously described scale-up issues with PICUP limits the utility of this approach. Moreover, batch to batch reproducibility for PICUP is limited by the aforementioned sensitivity of the method to parameters such as input illumination intensity and very short reaction times. For the instant invention, chemical cross-linking using DFDNB has definite advantages with regard to the ability to scale the process toward production of bulk quantities of material due to improved batch-to-batch consistency, lower level of monomeric contamination and a lower stringency of preparation conditions, such as necessary reaction times.

Through cross-linking with DFDNB, the oligomeric species generated are stabilized so that they are unable to revert back to monomers under standard physiological conditions. Those of ordinary skill in the art know that manipulation of reaction conditions in a chemical cross-linking reaction can generate differential ranges of Mw forms. For example, higher mass species can be favored by extending reaction time, increasing the molar concentration of cross-linking reagent relative to substrate, or increasing reaction temperature. An important aspect of the claimed invention is the ability to separate these species by SEC and generate an oligomeric pool which is devoid of monomer. This is an important consideration since, as previously described, extending the cross-linking reaction to completely convert monomer to oligomer will result in a high proportion of material outside the desired mass range, which would in effect lower the yield of desired species.

It is an established observation that short polypeptide sequences are generally poorly antigenic when used alone as immunogens (Perelson, A. S, and Wiegel, F. W., Fed. Proc. 40: 1479-1483 (1981); Dintzis, R. Z., et al., J. Immunol., 143:1239-1244 (1989)). The ability of such peptides to induce high titer immune responses can be markedly enhanced by either cross-linking the monomeric species or by conjugation, i.e. covalently coupling the peptide to a larger biomolecule, which in most cases is a carrier protein. Thus, the cross-linked amyloid-derived peptides of the present invention are expected to provide immunogens with such enhanced immunogenicity.

Literature reports suggest that native amyloid peptides are only weakly immunogenic given the observation that large amounts of antigen need to be administered multiple times in order to overcome the tolerance barrier to a self antigen and induce a response (Lambert, M. P., et al, J. Neurochem. 79: 595-605 (2001); Kayed, R., et al., Science 300: 486-489 (2003)). Moreover, even between species such as mouse and human, the similarity between Aβ sequences prevents high titer IgG-dominant responses to the human peptide when administered in mice. These low antibody responses are undesirable for identifying therapeutic monoclonal antibodies insofar as the potential pool of antibodies having the correct affinity is significantly restricted when low IgG titers are induced. Moreover, since the desired response is that directed toward the oligomeric species, a strategy for presenting stabilized oligomers in the absence of monomer would provide the highest probability of achieving the desired response. The instant method presented herein would provide the desirable component for use in such an immunogen. A similar strategy can be envisioned for peptide-carrier conjugates if the initial formulation of activated peptide includes an oligomer-generation step. In the instant matter this is achieved by preparation of peptide according to ADDL-generation protocols.

Those skilled in the art would recognize certain disadvantages of immunizing with native preparations of amyloid peptides or ADDLs including, but not limited to, (1) the occurrence of poor immune responses due to the difficulty of overcoming self tolerance, (2) the potential for the deleterious effect of raising an immune response to a self antigen and (3) the lack of oligomer stability in such preparations. Although (1) and (2) may appear to be mutually exclusive, they are, in principle, related. As discussed above, cross-linking and/or conjugation to immune-enhancing carrier proteins can be used to enhance the response to the amyloid peptides. However, if the response is largely directed toward immuno-dominant epitopes which may be shared between soluble monomer and toxic oligomers, then no oligomer-specificity is gained and, in fact, autoimmune effects may be realized since the normal form of the peptide may now be recognized by the immune system. Without wishing to be bound by any theory, inasmuch as the oligomer ADDL species is the acutely neurotoxic species associated with AD, antibodies specific to these forms should be able to overcome the disadvantages stated above and provide a safer means to induce an immune response and/or to administer. The instant invention provides a means to stabilize and isolate these oligomeric species for presentation as a more specific immunogen.

### EXAMPLES

### Example 1

### Preparation of Aβ-Derived Peptides

Peptide sequences (SEQ ID NOS: 1 and 2) were obtained from American Peptide Company (Sunnyvale, Calif.) or were synthesized in-house (SEQ ID NOS: 3-7). For in-house synthesized Aβ derived peptides, peptides were prepared by solid-phase synthesis on an Applied Biosystems automated peptide synthesizer using Fmoc chemistry protocols as supplied by the manufacturer (PE Biosystems, Foster City, Calif.). Following assembly the resin bound peptide was deprotected and cleaved from the resin using a cocktail of 94.5% trifluoroacetic acid, 2.5% 1,2-ethanedithiol, 1% triisopropylsilane and 2.5% H₂O. Following a two hour treatment the reaction was filtered, concentrated and the resulting oil triturated with ethyl ether. The solid product was filtered, dissolved in 50% acetic acid/H₂O and freeze-dried. Purification of the semi-pure product was achieved by RPHPLC using a 0.1% TFA/H₂O/acetonitrile gradient on a C-18 support Fractions were evaluated by analytical HPLC. Pure fractions (>98%) were pooled and freeze-dried. Identity was confirmed by amino acid analysis and mass spectral analysis.

### Example 2

### Chemical Cross-Linking of Aβ₄₂ Peptide

This example presents the chemical cross linking of Aβ₄₂ (SEQ ID NO. 2). The peptide was removed from storage at −70° C. and allowed to equilibrate to room temperature. Twenty two mg of the peptide powder was weighed into a polypropylene tube and solubilized with 2.2 mL of 1,1,1,3,3,3 hexafluoro-2-propanol (HFIP). The peptide solution was incubated in a 37° C. water bath for one hour and then sub-aliquoted into 2 mg (200 μl) retains. The peptide retains were frozen using a thy ice/ethanol bath and placed into a SpeedVac concentrator (Thermo-Electron Corporation, West Palm Beach, Fla.) to evaporate the solvent from the peptide. The dry peptide was stored at −70° C.

Four-2 mg retains (8 mg total) of Aβ ₄₂ were thawed. To each retain, was added 1.25 mL of 25 mM sodium borate, pH 8.5 buffer and tubes were placed on a vortex mixer at low speed to shake for 10-15 minutes. The solubilized peptides were pooled and an additional 5.4 mL of borate buffer was added to bring total volume to 10.4 mL (0.77 mg peptide/mL). The solution was mixed by inversion and 1.0 ml of 20 mM 1,5-difluoro-2,4-dinitrobenzene (DFDBN) (in 50% ethanol/50% water) was added and the solution mixed by inversion again. The cross linking reaction proceeded at room temperature for 10.5 to 12.5 minutes. The reaction was quenched by adding 300 μL of 50 mM dithiothreitol.

Alternatively, the reaction was used to prepare a cross-linked species of an oligomeric ADDL preparation. HFIP-dried peptide (Aβ₄₂) (8 mg) was solubilized in 354.4 μL, of DMSO and this solution was gradually added to 10.04 mL of 25 mM sodium borate buffer, pH 8.5 with intermittent vortexing. The peptide solution was incubated at 4° C. for 24 hours to form ADDLs and then cross linked as described above.

### Example 3

### Size Exclusion Chromatographic (SEC) Separation of Cross-Linked Aβ₄₂ Peptide

This example presents the chromatographic separation of the cross-linked oligomers of Aβ₄₂ of different molecular weights. The chromatography was performed on an Alliance 2690 Separations Module (Waters Corp., Milford Mass.) coupled to a 996 photodiode array detector (Waters Corp., Milford Mass.). The size exclusion column was a TSK gel G3000 PW (21.5 mm×60 cm) with a TSK PWH guard column (21.5 mm×7.5 cm) (Tosoh Bioscience, Montgomeryville, Pa.) placed in line before the SEC column. The SEC column was heated to 45-48° C. using Thermolyne flexible electric heating tape (Barnstead International, Dubuque IO) wrapped around the column and controlled by a Thermolyne type 45500 input controller (Bamstead International, Dubuque IO). The temperature was monitored by a digital thermometer with a flexible temperature sensor (Fisher Scientific, Pittsburgh, Pa.). Chromatography was performed on the cross-linked peptide immediately after the reaction was quenched. The cross-linked peptide was loaded onto the column through an empty 12 mL sample loop using a manual injector. The mobile phase consisted of a 1:1 mixture of acetonitrile and 25 mM tricine, 150 mM NaCl, pH 8.5 buffer and was pumped at a rate of 1.5 mL/minute. Through the process of method development it was observed that both elevated temperature and the use of an aqueous:organic mobile phase were critical for efficient removal of monomeric peptide from the final pool. Absorbance was monitored at 215 and 280 nm. Fractions were collected in 13×100 mm glass tubes using a Frac-100 fraction collector (Amersham Bioscience, Piscataway, N.J.) at a rate of 1 fraction/minute. DMSO (15 μL) was added to each fraction and the fractions were vortexed. The fractions were placed in a SpeedVac concentrator (Themo-Electron Corporation, West Palm Beach, Fla.) for 2 to 3 minutes to remove acetonitrile and were then stored at 4° C. Denaturing/nonreducing SDS-PAGE using 10-20% tricine gels was performed on the SEC fractions. The gels were stained using a Silver Xpress staining kit (Invitrogen, Carlsbad, Calif.). Fractions containing oligomers of the mass range 100 kDa to 200 kDa and very little monomer by gel were pooled. Remaining acetonitrile was removed using a stream of N₂ gas. Protein concentration was determined using either a commercial Bradford or bicinchoninic acid (BCA) assay kit (Pierce Biotechnology, Rockford, Ill.).

### Example 4

### Formulation of Cross-Linked Product for Animal Immunizations and Immunogenicity Studies

This example presents the formulation of a product for animal immunizations and immunogenicity studies. The SEC pool of high molecular weight oligomers of chemically cross linked Aβ₄₂ peptide was formulated into 25 mM tricine, 150 mM NaCl, pH 8.5 buffer to a final protein concentration of 200 μg/mL. Additionally, the SEC pool was formulated as stated above and then aluminum hydroxide was added with gentle vortex to a final alum concentration of 450 μg/mL. Formulations were prepared fresh for each immunization and stored at 2-8° C. prior to injection.

Studies were initiated to evaluate the immunogenicity of the chemically cross-linked Aβ₄₂ peptide in mice as well as to develop Aβ₄₂ peptide-specific monoclonal antibodies. Female Bable/c mice, 10 per group, were immunized intramuscularly with 20 mcg of cross-linked Aβ₄₂ antigen, formulated either in Alum or in Freund's adjuvant. The immunization involved a total of five injections in four week intervals. Blood samples were collected two weeks after injection and will be determined for antibody titers against Aβ₄₂ antigen. For monoclonal antibody production, splenocytes will be isolated from the immunized animals and they will be fused with SP2/0 myeloma cells by standard procedures. The resulting hybridomas will be screened for the production of specific monoclonal antibodies by Enzyme-linked immunosorbant assay (ELISA).

### Example 5

### Chemical Conjugation of Aβ Peptides to OMPC

This example represents the chemical conjugation of peptides derived from human Aβ (1-42) to purified outer membrane protein complex (OMPC) of Neisseria meningitidis, type B. The chemical nature of the coupling is a reaction between maleimide-derivatized peptide and thiol-derivatized protein of the membrane complex. Modified amyloid peptides described above (SEQ ID NOS: 6 and 7) were synthesized as described and used for conjugation. These peptides contained an inversion of amino acid residues 1-8 followed by the native 9-42 amyloid precursor protein sequence for the purpose of directing the immune response away from the immunodominant 1-10 amino acid residue region. For N-terminal peptides, the maleimide functionality was attached directly to the primary amine of the Aha spacer while for C-terminal attachment it was placed on the s-amine of a terminal lysine residue. All manipulation of OMPC-containing solutions was performed in a laminar flow environment following standard aseptic techniques.

A. Thiolation of OMPC

Purified, sterile OMPC was obtained from Merck Manufacturing Division and was thiolated on a portion of its surface-accessible lysine residues using the reagent N-acetylhomo-cysteinethiolactone (NAHT, Aldrich, St. Louis, Mo.). OMPC in water, 117 mg, was pelleted by centrifugation at 289,000×g for 60 min at 4° C. and the supernatant was discarded. N₂-sparged activation buffer (0.11 M sodium borate, pH 11) was added to the centrifuge tube and the pellet was dislodged with a glass stir rod. The suspension was transferred to a glass Dounce homogenizer and resuspended with 30 strokes. The centrifuge tube was washed and the wash dounced with 30 strokes. Resuspended pellet and wash were combined in a clean vessel to give a OMPC concentration of 10 mg/mL. Solid DTT and EDTA were dissolved in N₂-sparged activation buffer and charged to the reaction vessel at a ratio of 0.106 mg DTT/mg OMPC and 0.57 mg EDTA/mg OMPC. After gentle mixing, NAHT was dissolved in N₂-sparged water and charged to the reaction at the ratio of 0.89 mg NAHT/mg OMPC. The reaction proceeded for three hours at ambient temperature, protected from light. At completion, OMPC was pelleted as described above and resuspended at 6 mg/mL by Dounce homogenization in N₂-sparged conjugation buffer (25 mM sodium borate, pH 8.5, 0.15 M NaCl) to wash the pellet. For final re-suspension, the OMPC was pelleted as above and re-suspended at 10 mg/mL by Dounce homogenization in N₂-sparged conjugation buffer. A final low-speed centrifugation was performed at 1,000×g for 5 min at 4° C. to remove any aggregated product. An aliquot was removed for free thiol determination by Ellman assay and the bulk product was stored on ice in dark until use. Measured thiol content was between 0.2 to 0.3 μmol/mL.

B. Conjugation of Peptide to OMPC

Functional maleimide content of peptides was assumed to be 1:1 on a molar basis. Sufficient peptide was weighed to give an equimolar amount of maleimide to total thiol. The targeted total OMPC protein for each conjugation was about 15 mg. Peptides were resuspended in DMSO at 20 mg/mL and diluted to 5 mg/mL in 25 mM sodium borate, pH 8.5, 0.15 M NaCl. Peptide solutions were slowly added to thiolated OMPC solution while gently vortexing. The reactions were protected from light and incubated at ambient temperature without mixing for 14 hours. Residual free OMPC thiol groups were quenched with a 2-fold molar excess of N-ethylmaleimide for three hours at ambient temperature. A thiolated OMPC-only control was carried through the conjugation protocol in parallel. Upon completion of quenching, conjugate and control were transferred to 100,000 Da molecular weight cut-off dialysis units and dialyzed exhaustively against at least 5 changes of 20 mM sodium borate, pH 8.5 buffer. Upon completion of dialysis, samples were transferred to 15 ml polypropylene centrifuge tubes and centrifuged at 1,000×g for 5 min at 4° C. to remove any aggregated material. Aliquots were removed for analysis and the bulk was stored at 4° C.

C. Analysis

Total protein was determined by the modified Lowry assay and samples of conjugate and controls were analyzed by quantitative amino acid analysis (AAA). Peptide to OMPC molar ratios were determined from quantitation of the unique residue S-dicarboxyethylhomocysteine (SDCEHC) which was released upon acid hydrolysis of the nascent peptide-OMPC bond. The OMPC-specific concentration was determined from hydrolysis-stable residues which were absent from the peptide sequence and thus unique to OMPC protein. Assuming 1 mol of peptide for every mol SDCEHC, the ratio of SDCEHC/OMPC was thus equivalent to the peptide/OMPC content. The mass loading of peptide could be calculated from this ratio using the peptide molecular weight and an average OMPC mass of 40,000,000 Da.

The covalent nature of the conjugation was qualitatively confirmed by SDS-PAGE analysis using 4-20% Tris-glycine gels (Invitrogen, Carlsbad, Calif.) where an upward shift in mobility was observed for the Coomassie-stained conjugate bands relative to control.

### Example 6

### Assay to Detect Cross Linked Aβ Oligomers

This example represents an assay to detect cross linked Aβ oligomers produced by the method claimed herein. The assay described below is described in a concurrently filed application by Ming Tain Lai et al., U.S. Ser. No. 60/695,527 and incorporated herein as if set forth at length.

A. Preparation of ELISA Plates

Corning-Costar ELISA plates are coated by the addition of 100 μl of 5 μg/ml of the 6E10 antibody (Signet Labs, Dedham, M A) (stock=1 mg/ml) in a coating buffer (50 mM Na-bicarbonate, pH9.6) to each well on a 96 well plate. The resulting plates were covered with a thin adhesive film to prevent evaporation and loss of sample volume and then slowly shaken overnight at 4° C. The plates were washed twice with PBS-T (0.1% Tween-20 in regular PBS) the following day. The wells were then blocked with 200 μl of SuperBlock with PBS for at least one hour.

B. Assay Protocol

An Aβ cross linked oligomer standard, a Aβ₄₂ monomer control and the samples (100 μL) to be evaluated are added to the pre-coated plates followed by the addition of 50 μl of 6E10-AP (1:500 dilution in 0.3% Tween-in SuperBlock) to all samples. The resulting plates are incubated at 4° C. overnight with shaking and washed 5× with 200 μl PBS-T the following day. The alkaline phosphate substrate (100 μL) is introduced to each well on the plate to initiate the reaction. After incubation at room temperature (RT) for 30 minutes, the samples are read on a standard multimode reader with luminescence detection capability (formerly Analyst, LJL BioSystems, Inc, Sunnyvale, Calif.; presently Molecular Devices Corporation, Analyst GT multimode reader, Sunnyvale, Calif.). Based on a fit of the standard curve data to an appropriate model (either linear or quadratic fit in Microsoft Excel or 3^{rd} order spline fit in IMP(SAS Institute, Cary, N.C.), the cross linked Aβ oligomer concentration of each sample is calculated. Values that were statistically meaningfully above background or monomer control levels were viewed as cross linked Aβ oligomer related signals.

While this assay is illustrated using an ELISA detection format, those skilled in the art would recognize that this assay could be carried out using an ECL or ALPHA screen format as set forth in Ming Tain Lai et al. above.

What is claimed is:

1. A method for producing a covalently stabilized antigen comprising:
a. preparing a solution of solubilized Aβ-derived peptide;
b. combining said solution with a near-zero length bifunctional cross-linking agent to form a mixture;
c. incubating said mixture at a temperature for a period of time sufficient to effect cross-linking; and
d. providing the cross-linked product of step (c) to be used as an antigen.

2. The method of claim 1 wherein the bifunctional cross-linking agent is 1,5-difluoro-2,4-dinitrobenzene (DFDNB).

3. The method of claim 2 wherein the cross-linked product has a mass range of 100 kDa to 200 kDa.

4. The method of claim 2 wherein the cross-linked product has monomer contamination of less than 5%.

5. The method of claim 2 wherein the cross-linked product is additionally formulated with an adjuvant to be used as an immunogen.

6. The method of claim 5 wherein said adjuvant is a saponin-based adjuvant.

7. The method of claim 5 where said formulation is used as a pharmaceutical composition.

8. A method of producing a covalently stabilized antigen having a conformational epitope of Aβ comprising:
a. preparing a solution of solubilized Aβ-derived peptide;
b. combining said solution with a bifunctional cross-linking agent to form a mixture;
c. incubating said mixture at a temperature for a period of time sufficient to effect cross-linking; and
d. providing the cross-linked product of step (c) to be used as an antigen.

9. The method of claim 8 wherein the bifunctional cross-linking agent is 1,5-difluoro-2,4-dinitrobenzene (DFDNB).

10. The method of claim 9 wherein the cross-linked product has a mass range of 100 kDa to 200 kDa.

11. The method of claim 9 wherein the cross-linked product has monomer contamination of less than 5%.

12. The method of claim 9 wherein the cross-linked product is additionally formulated with an adjuvant to be used as an immunogen.

13. The method of claim 12 wherein said adjuvant is a saponin-based adjuvant.

14. The method of claim 12 where said formulation is used as a pharmaceutical composition.

15. A method for producing a purified covalently stabilized antigen comprising:
a. preparing a solution of solubilized Aβ-derived peptide;
b. combining said solution with a bifunctional cross-linking agent to form a mixture;
c. incubating said mixture at a temperature for a period of time sufficient to effect cross-linking;
d. purifying the plurality of cross-linked products from step (c) using chromatographic separation based on mass of said cross-linked products; and
e. providing the purified product of step (d) to be used as an antigen.

16. The method of claim 15 wherein the bifunctional cross-linking agent is 1,5-difluoro-2,4-dinitrobenzene (DFDNB).

17. The method of claim 15 wherein the chromatographic separation of step (d) further comprises a 1:1 mixture of acetonitrile and an aqueous buffer and a column temperature of 45° C. to 48° C.

18. The method of claim 15 wherein the cross-linked product has a mass range of 100 kDa to 200 kDa.

19. The method of claim 15 wherein the cross-linked product has monomer contamination of less than 5%.

20. The method of claim 15 wherein the cross-linked product is additionally formulated with an adjuvant to be used as an immunogen.

21. The method of claim 20 wherein said adjuvant is a saponin-based adjuvant.

22. The method of claim 20 where said formulation is used as a pharmaceutical composition.


SEQUENCE LISTING (7 sequences)

SEQ ID NO: 1 - protein, 40 aa, Homo sapien
DAEFRHDSGYEVHHQKLVFFAEDVGSNKGAIIGLMVGGVV

SEQ ID NO: 2 - protein, 42 aa, Homo sapien [sequence omitted]

SEQ ID NO: 3 - protein, 42 aa, Homo sapien [sequence omitted]

SEQ ID NO: 4 - protein, 34 aa, Homo sapien
GYEVHHQKLVFFAEDVGSNKGAIIGLMVGGVVIA

SEQ ID NO: 5 - protein, 34 aa, Homo sapien; identical to SEQ ID NO: 4

SEQ ID NO: 6 - protein, 42 aa, Homo sapien
SDHRFEADGYEVHHQKLVFFAEDVGSNKGAIIGLMVGGVVIA

SEQ ID NO: 7 - protein, 42 aa, Homo sapien; identical to SEQ ID NO: 6